(19) United States
(12) Patent Application Publication
(10) Pub. No.: US 2025/0326076 A1
(43) Pub. Date: Oct. 23, 2025

(54) SYSTEMS AND METHODS FOR HYBRID AUTONOMOUS MANUFACTURING

(71) Applicant: Ohio State Innovation Foundation, Columbus, OH (US)

(72) Inventors: Glenn Steven DAEHN, Columbus, OH (US); Michael Anthony GROEBER, Columbus, OH (US); Stephen R. NIEZGODA, Columbus, OH (US); Nathan D. AMES, Sunbury, OH (US); Howard David DEAN, Upper Arlington, OH (US); Brian Patrick THURSTON, Columbus, OH (US); Jian CAO, Evanston, IL (US); Wei CHEN, Wilmette, IL (US); Tony LaVaun SCHMITZ, Knoxville, TN (US); John Joseph Francis LEWANDOWSKI, Shaker Heights, OH (US)

(21) Appl. No.: 19/100,237

(22) PCT Filed: Aug. 9, 2023
(86) PCT No.: PCT/US2023/029817

§ 371 Date: Jan. 31, 2025

Related U.S. Application Data
(60) Provisional application No. 63/396,386, filed on Aug. 9, 2022.
(60) Provisional application No. 63/416,822, filed on Oct. 17, 2022.

(51) Int. Cl. B23P 21/00; B33Y 10/00; B33Y 30/00; B33Y 50/02

(52) U.S. Cl. CPC B23P 21/004; B33Y 10/00; B33Y 30/00; B33Y 50/02

(57) ABSTRACT

A component manufacturing device called Auto-Fab (100) is disclosed. Each Auto-Fab system is a self-contained device including a housing (150), tools (120), robotics (110), sensors (113), and computing functionality (115) that is configured to manufacture a variety of components (160) using various materials available at a location of the Auto-Fab. The Auto-Fab, using the robotics and tools, may be programed to autonomously perform a variety of manufacturing techniques including, but not limited to, deformation, casting, machining, and welding The manufacturing processes used by the Auto-Fab for a particular component may be designed using an iterative feedback method (200; 300) where the manufacturing processes are continuously tweaked and tuned based on a comparison of a manufactured component with predicted attributes.

### CROSS-REFERENCE TO RELATED APPLICATIONS

This application claims priority to U.S. Provisional Patent No. 63/396,386, filed on Aug. 9, 2022, entitled “DEVICES, SYSTEMS, AND METHODS FOR HYBRID AUTONOMOUS MANUFACTURING.” This application further claims priority to U.S. Provisional Patent No. 63/416,822, filed on Oct. 17, 2022, entitled “DEVICES, SYSTEMS, AND METHODS FOR HYBRID AUTONOMOUS MANUFACTURING.” The contents of both are hereby incorporated by reference.

### BACKGROUND

Manufacturing in space and other harsh or austere locations presents several significant challenges that make the process inherently difficult. First and foremost, the absence of a stable environment poses a major obstacle. Space, for instance, lacks gravity, which affects various manufacturing processes such as fluid dynamics, solidification, and particle behavior. Without gravity, the separation and settling of particles become problematic, making it challenging to achieve uniformity and consistency in manufacturing products. Similarly, extreme temperatures, vacuum conditions, and radiation levels in space and other harsh environments can adversely affect materials, equipment, and overall manufacturing processes, requiring specialized technologies and designs to mitigate these effects. Further it is quite costly to bring manufacturing equipment to space, so smaller systems are favored. Lastly production volumes may be smaller. All these factors favor highly-controlled, lightweight and agile systems over systems that are currently common.

Secondly, the logistical complexities associated with manufacturing in space or other harsh locations contribute to the difficulty of the process. Transporting raw materials, equipment, and personnel to these remote and inhospitable locations is a daunting task. Launching and delivering supplies to space can be costly, time-consuming, and risky. The need for regular resupply missions further adds to the overall complexity and expense of manufacturing operations. In addition, the lack of a readily available infrastructure and support systems in these locations necessitates the development of self-sufficient manufacturing facilities, requiring advanced automation, robotics, and efficient resource management.

### SUMMARY

A component manufacturing device called an autonomous factory artisan box (“Auto-Fab”) is disclosed. Each Auto-Fab system is a self-contained device including a housing, tools, robotics, sensors, and computing functionality that is configured to manufacture a variety of components using various materials available at a location of the Auto-Fab. An Auto-Fab can be a physical volume or a virtual space where similar functions are connected by logistics using the control algorithms disclosed here. The Auto-Fab, using the robotics and tools, may be programed to autonomously perform a variety of manufacturing techniques including, but not limited to, deformation, casting, machining, and welding. The physical Auto-Fab may be moveable and may be placed in a variety of locations to bring autonomous manufacturing capabilities to locations where such capabilities were previously unavailable (e.g., outer space and other hostile environments). The manufacturing processes used by the Auto-Fab for a particular component may be designed using an iterative feedback loop where the manufacturing processes are continuously tweaked and tuned based on a comparison of a manufactured component with desired tolerances and design parameters.

In one aspect, a method to concurrently design a component (that conforms to engineering requirements such as strength, compliance, fracture resistance, environmental resistance and so on) and a manufacturing process for that component is provided. Design in this context may include evaluating many options and finding the performance for these options. The total number of options may become immense. Example options include, but are not limited to, deformation processing; machining (material removal), additive manufacturing, casting, welding, and any combinations of the above. Further multiple different types of materials may be used in the design and additive manufacturing offers the possibility of graded composition, which may be useful and further improved by deformation processing.

The component design and manufacturing process may adhere to one or more performance metrics. These performance metrics may include, but are not limited to, cost, strength stiffness (engineered in varied directions), fracture resistance, fatigue resistance, corrosion resistance, and wear resistance.

To determine the component design and manufacturing process, a Pareto surface may be found by plotting or considering in 2-D or higher dimensional space the combination of metrics for each combination of design and manufacturing options. As a nearly infinite number of options exist, this will almost be a cloud with a surface. In each case, the performance for a given design and manufacturing option is estimated or measured. Comparisons between measured performance with known values of design or manufacturing attributes may be used to train or validate machine learning models, using methods that are standard in this field.

Automated or autonomous execution of the selected manufacturing process is imagined (e.g., using the Auto-Fab). For example, numerically controlled machining, additive manufacturing, and welding exists. These processes are initially automated, and in parallel numerical simulations may be done. Data from sensors is collected during the process. This data may be used to make in-process corrections using control algorithms. As data is collected, the control algorithms become more and more robust leading towards autonomous machine control.

In some embodiments, the design of the manufacturing process may include the order or sequence in which the operations are performed. For example, in one process welding may be followed by machining, and vice versa. This sequence of operations is part of the design. Additively manufactured components may be dramatically improved by deformation, and porosity can be reduced and made anisotropic to dramatically improve strength and fracture toughness or a resulting component.

In some embodiments, the transition from one process to another is an a part of the invention. Dimensional and local material state information may be aligned with the component being fabricated using standard transferable component bases or chucks. These chucks may have spring-loaded detents or other breakaway features, or may use springs or compliance to prevent overload during forging or deformation processing.

In some embodiments, the collection and use of data may allow model-based validation and certification of components. Data will be collected during the process and performance data will be collected afterward. This data will be used to train and validate machine learning models. As the models become more trusted and tested with more data we will be able to design parts and processes that rely on these data.

In some aspects, the techniques described herein relate to a method for manufacturing a component including: receiving design constraints for a component to be manufactured by a computing device; determining available tools and available materials at a location by the computing device; determining a design for the component based on the design constraints, determined available tools, and determined available materials by the computing device; determining a manufacturing process for the for the for the component based on the determined design and determined available tools by the computing device; executing the manufacturing process to generate the component by the computing device; receiving data generated during the manufacturing process about the generated component by the computing device; and based on the data generated during the manufacturing process, determining that the generated component does not satisfy the received design constraints by the computing device; and in response to the determination that the generated component does not satisfy the received design constraints, adjusting the determined manufacturing process by the computing device.

In some aspects, the techniques described herein relate to a method, further including in response to the determination that the generated component does not satisfy the received design constraints, adjusting the determined design by the computing device.

In some aspects, the techniques described herein relate to a method, wherein the manufacturing process is an automated manufacturing process and includes one or more of deformation, casting, machining, and welding.

In some aspects, the techniques described herein relate to a method, wherein the manufacturing process is performed by an Auto-Fab system, and the location is the location of the Auto-Fab system.

In some aspects, the techniques described herein relate to a method, wherein the Auto-Fab system includes a housing, the determined available tools, and a robotics component adapted to perform the manufacturing process using the determined available tools.

In some aspects, the techniques described herein relate to a method, wherein the component is a medical device.

In some aspects, the techniques described herein relate to a method, wherein the design constraints include geometric constraints, forces to be resisted by the component, and environment constraints.

In some aspects, the techniques described herein relate to a system including: at least one computing device; and a computer-readable medium storing computer-executable instructions stored thereon that when executed by the at least one computing device, cause the at least one computing device to: receive design constraints for a component to be manufactured; determine available tools and available materials at a location; determine a design for the component based on the design constraints, determined available tools, and determined available materials; determine a manufacturing process for the for the for the component based on the determined design and determined available tools; execute the manufacturing process to generate the component; receive data generated during the manufacturing process about the generated component; and based on the data generated during the manufacturing process, determine that the generated component does not satisfy the received design constraints; and in response to the determination that the generated component does not satisfy the received design constraints, adjust the determined manufacturing process. This process may be enhanced by modern computer science methods including capturing large data sets on both the component manufacturing process as well as component performance data with analysis by artificial intelligence techniques including training neural networks and using deep learning methods.

In some aspects, the techniques described herein relate to a system, further including computer-executable instructions stored thereon that when executed by the at least one computing device, cause the at least one computing device to: in response to the determination that the generated component does not satisfy the received design constraints, adjust the determined design by the computing device.

In some aspects, the techniques described herein relate to a system, wherein the manufacturing process is an automated manufacturing process and includes one or more of deformation, casting, machining, and welding.

In some aspects, the techniques described herein relate to a system, wherein the manufacturing process is performed by an Auto-Fab system, and the location is the location of the Auto-Fab system.

In some aspects, the techniques described herein relate to a system, wherein the Auto-Fab system includes a housing, the determined available tools, and a robotics component adapted to perform the manufacturing process using the determined available tools.

In some aspects, the techniques described herein relate to a system, wherein the component is a medical device.

In some aspects, the techniques described herein relate to a system, wherein the design constraints include geometric constraints, forces to be resisted by the component, and environment constraints.

In some aspects, the techniques described herein relate to a system, wherein data captured during the manufacturing process is used to test predictions from one or more machine learning or physics-based models to improve predictions.

In some aspects, the techniques described herein relate to a system, wherein data captured regarding manufactured components are used to test predictions from one or more machine learning or physics-based models to improve predictions.

In some aspects, the techniques described herein relate to a system, wherein the one or more machine learning or physics-based models are used to certify the manufactured components for use in high-performance applications and industries such as aerospace.

In some aspects, the techniques described herein relate to a non-transitory computer-readable medium storing computer-executable instructions stored thereon that when executed by at least one computing device, cause the at least one computing device to: receive design constraints for a component to be manufactured; determine available tools and available materials at a location; determine a design for the component based on the design constraints, determined available tools, and determined available materials; determine a manufacturing process for the for the for the component based on the determined design and determined available tools; execute the manufacturing process to generate the component; receive data generated during the manufacturing process about the generated component; and based on the data generated during the manufacturing process, determine that the generated component does not satisfy the received design constraints; and in response to the determination that the generated component does not satisfy the received design constraints, adjust the determined manufacturing process.

In some aspects, the techniques described herein relate to a non-transitory computer-readable medium, further including computer-executable instructions that when executed by the at least one computing device, cause the at least one computing device to: in response to the determination that the generated component does not satisfy the received design constraints, adjust the determined design by the computing device.

In some aspects, the techniques described herein relate to a non-transitory computer-readable medium, wherein the manufacturing process is an automated manufacturing process and includes one or more of deformation, casting, machining, and welding.

In some aspects, the techniques described herein relate to a non-transitory computer-readable medium, wherein the manufacturing process is performed by an Auto-Fab system, and the location is the location of the Auto-Fab system.

In some aspects, the techniques described herein relate to a non-transitory computer-readable medium, wherein the Auto-Fab system includes a housing, the determined available tools, and a robotics component adapted to perform the manufacturing process using the determined available tools.

In some aspects, the techniques described herein relate to a non-transitory computer-readable medium, wherein the component is a medical device.

In one aspect, a method to concurrently design a component and a manufacturing process for that component is provided. The method may include: concurrently designing part and process with predictions of key performance attributes; in design, considering all reasonable combinations of component topology and manufacturing processes (recognizing that processes may be hybrid); predicting performance attributes (cost, stiffness, strength, etc.) for each possible combination; selecting one for more designs from the Pareto surface and making it, using hybrid autonomous methods; collecting data during the process, and performance data for the components that are created; and using this data to train models used to design the components and manufacturing processes.

In one aspect, a method to concurrently design a component and a manufacturing process for that component is provided. As a first step, the problem is defined. The problem definition may include a component to be created and characteristics of the component such as loads, environment, geometric constraints, lifetime of the component failure risks, applied loads, and a number of parts required to build the component.

As a second step, multiple possible designs for the component are created. These designs may include different possible materials and shapes for the component, as well as different possible manufacturing processes for the component.

As a third step, attributes are predicted for each possible design using one or more models. These attributes include cost, strength, availability, fracture resistance, durability, lead time, and supply chain resistance.

As a fourth step, a best design and manufacturing process are selected. The best design and manufacturing process may be selected using Pareto techniques, for example.

As a fifth step, the selected best design is created by an Auto-Fab according to the selected best process. Sensors may collect data during the manufacturing process that may be used for control and later analysis.

As a sixth step, the generated component is tested. The tests may measure the attributes of the manufactured component and to determine any defects or other unexpected results.

As a seventh step, the models used to predict the attributes are updated based on the tests. In this way the models are improved and will better predict future attributes. The steps of two through seven may be repeated overtime to generate better components and improve the attribute prediction models.

As an eighth step, the models are used to certify components. The continuously updated models may be used to predict the performance of components and manufacturing processes and to certify the components and processes as suitable for various applications.

### BRIEF DESCRIPTION OF THE DRAWINGS

The components in the drawings are not necessarily to scale relative to each other. Like reference numerals designate corresponding parts throughout the several views.

FIG. 1 is an illustration of an example of an Auto-Fab system;

FIG. 2 is an illustration of an example method for designing a component and a manufacturing process;

FIG. 3 is an illustration of another example method for designing a component and a manufacturing process; and

FIG. 4 illustrates an example computing device.

### DETAILED DESCRIPTION

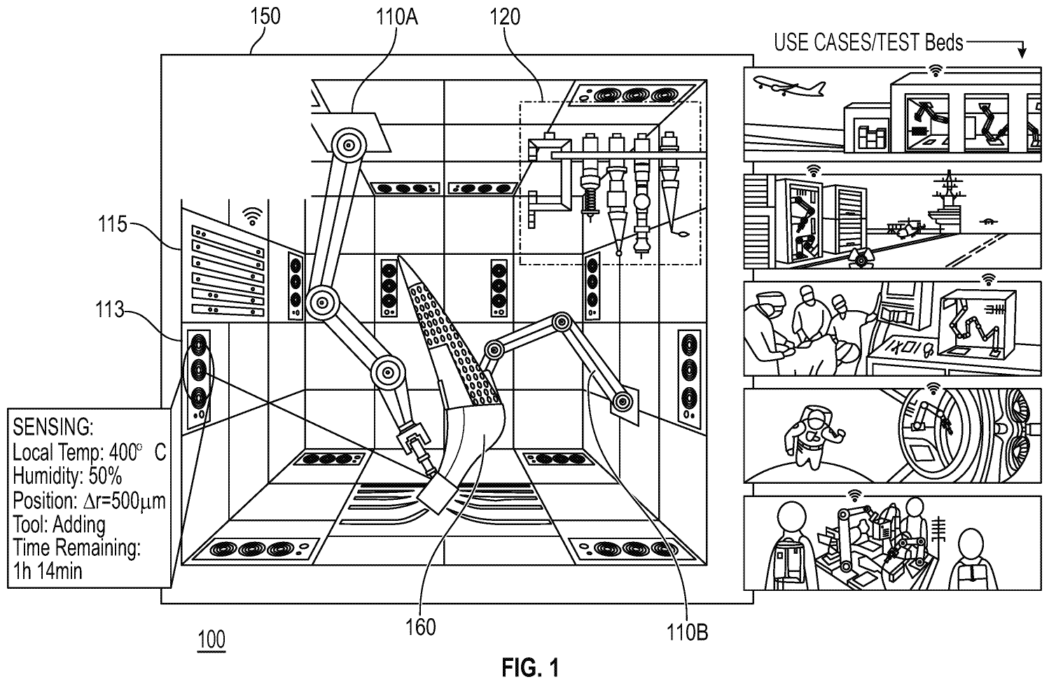

FIG. 1 is an illustration of an example autonomous fabrication (“Auto-Fab”) system 100. In the example shown, the Auto-Fab system 100 includes several components including a robotics component 110, a tool component 120, a sensor component 113, and a computing component 115 all within a housing 150. More or fewer components may be supported.

The Auto-Fab 100 may be a self-contained autonomous manufacturing device that can be easily transported and placed directly into a variety of scenarios and use cases including, but not limited to, regional manufacturing centers, health care facilities, forward operating bases, outer space, and classrooms. Austere environments (those without larger manufacturing centers or easy logistics), near point of need are areas of where Auto-Fabs may be particularly valuable. The Auto-Fab system 100 may allow for the development and transitioning of new manufacturing technologies; the educating and training of a manufacturing workforce, and the expansion the capabilities of the domestic manufacturing. The Auto-Fab system 100 integrates multiple discrete research fields in manufacturing, achieving capabilities similar to multi-skilled artisans (e.g., blacksmiths, tool and die makers), who can use small simple tools to fabricate a wide array of useful products. In the example shown in FIG. 1, the Auto-Fab 100 is being used to fabricate an artificial hip 160.

The housing 150 may be sized to accommodate the components 113, 115, and 120 as well as whatever components are to be created by the Auto-Fab 100 including the necessary raw materials. The housing 150 may take on a wide variety of sizes depending on the most common use cases. It could have a construction that may resemble a common machining center, fabricated from precision steel with accommodation for multiple fixtures, tool holders and sensors, for example.

The tools component 120 may include a variety of tools that may be used by the robotics component 110 to fabricate a component or part using available materials. Example tools that may be included in the tool component 120 include, but are not limited to: hydraulic C-frame presses with interchangeable end dies; inspection heads (ultrasonic, eddy current, visual, dimensional, LIDAR, etc., etc.); mechanical hammers with varied tips that may be pneumatically, hydraulically, or electromagnetically actuated; heating devices that can be used for welding or heat treatment (flame, laser, induction, etc.); deformation tools like english wheels, stretchers, shrinkers, etc.; deposition devices, paint, powdered metal to be sintered, weld addition; weld heads (arc, laser, flame, etc.); peening tools; high velocity impulse devices (laser, impact, vaporizing foil, micro explosive, etc.); material removal tools (files, grinders, saws, machining heads, etc.); and additive+X manufacturing tools. Other types of tools may be supported.

The robotics component 110 may include a plurality of robotic arms (e.g., the robotic arms 110A and 110B). Suitable robotic arms may have any type of topology including usual revolute systems, gantry cartesian systems with 3 to 6 degrees of freedom with respect to position and rotation. Ideal robots have high dimensional precision, and stiffness. In the case of metal forming large forces are not usually required. This can be developed by a separate press. There may be situations wherein human-safe robots are required for collaborative tasks. A variety of end-effectors may be used for holding, manufacturing, inspecting or human interaction. One or several robots may be used at once. Other types of robotics components 110 may be supported.

In some embodiments, the robotic arms 110 may include tool Interconnects such as standard robot end effectors that give both dimensional registry to the possible tools listed above, but also supply power, communications and possibly gasses or liquids to the tool. Standardized quick disconnects may be needed. Fixtures for gripping and transferring workpieces from one operation to another while maintaining dimensional integrity may also be included. These fixtures will have standardized interconnects and may have standard connections for electrical power, sensors, cooling water or other functionality. Note that for some operations, such as heat treating, it may be necessary to disconnect the workpiece and reconnect it or transfer it to a different cell either in the same building or remotely. Dimensional registry features on the tool and part may aid in re-establishing the part's coordinate system.

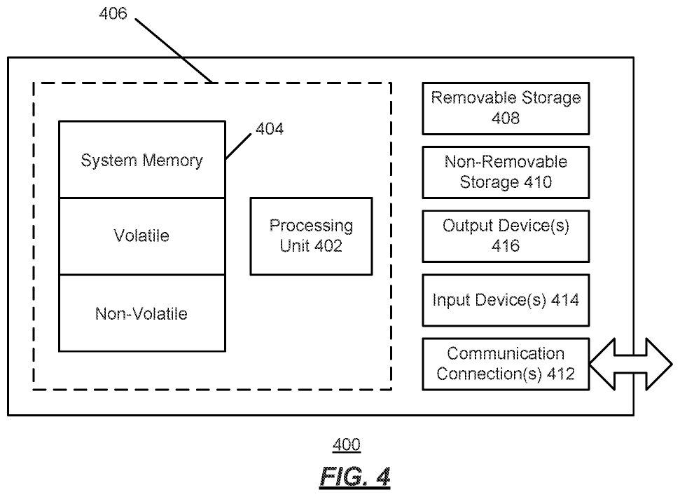

The computing component 115 may be implemented by one or more general purpose computing devices such as the computing system 400 illustrated with respect to FIG. 4. The computing component 115 may receive data from the sensor component 113 and the robotics component 110 and may control the operation of the sensor component 113 and the robotic component 110, as well as other functionality of the Auto-Fab 100.

The computing component 115 may include functions and algorithms for various functions and work-sequences that may be used by the Auto-Fab 100. The fabrication of many components may involve several discrete operations, such as shaping, welding and surface finishing. Depending upon the tools and processes used, there are multiple discrete sequences that may be stored and used by computing component 115 to fabricate a component.

In some embodiments, the computing component 115 may include functions and algorithms for control of particular operations that also are part of the larger scheme of component generation. For example, in painting algorithms used to paint a component there is a need for full coverage without dripping. In welding, many parameters are controlled with feedback to assure a good weld. These individual process control approaches can be algorithmic or based on machine learning and based on the observation of human artisans. Quality in these processes is assured by the computing component 115 understanding and predicting, with varied and possibly high certainty, the state of the material structure that is created or processed at each location within a component. In a prototypical application this is related to assuring that the material is subject to thermomechanical processing within known limits of temperature or strain to meet minimum required properties. There are far more advanced scenarios that use Integrated computational materials engineering with statistical limits from validation and verification exercises that include the accumulation of large data sets and the use of this data with machine learning algorithms to improve future processes. The learning in one Auto-Fab 100 may improve the algorithms used at other locations.

Sensor component 113 may include a variety of sensors and sensor types that may monitor and report conditions within the housing of the Auto-Fab 100 (e.g., temperature and humidity), conditions of the component being constructed (e.g., temperature and size), conditions of the robotics component 110 (e.g., temperature and force), and temperature of the tools component 120 (e.g., temperature, power, and material or resource levels). Example sensors include, but are not limited to, thermometers, accelerometers, scales, humidity sensors, pressor and force sensors, distance and proximity sensors, cameras, and infrared sensors. Other types of sensors may be supported. The information provided by the sensor component 113 to the computing component 115 may allow the computing component 115 to make adjustments to the manufacturing process while creating a particular component and to monitor the progress of the manufacturing process.

Of particular interest is sensing that reveals the state of the material in the material being manufactured. Thermomechanical processing allows the improving of material microstructure, which in turn improves material properties. Understanding the changes in shape, can allow one to infer strains, and the use of thermal cameras can allow estimation of thermal path. Tracking this strain-temperature path can allow the estimation of material microstructural development. It may also be of interest to track surface condition during machining or peening. This can enhance fatigue resistance.

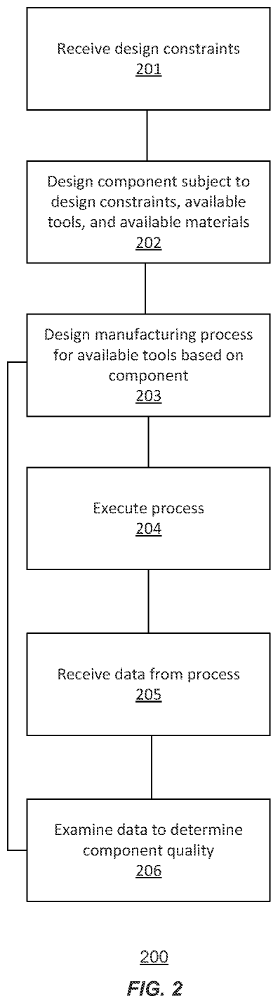

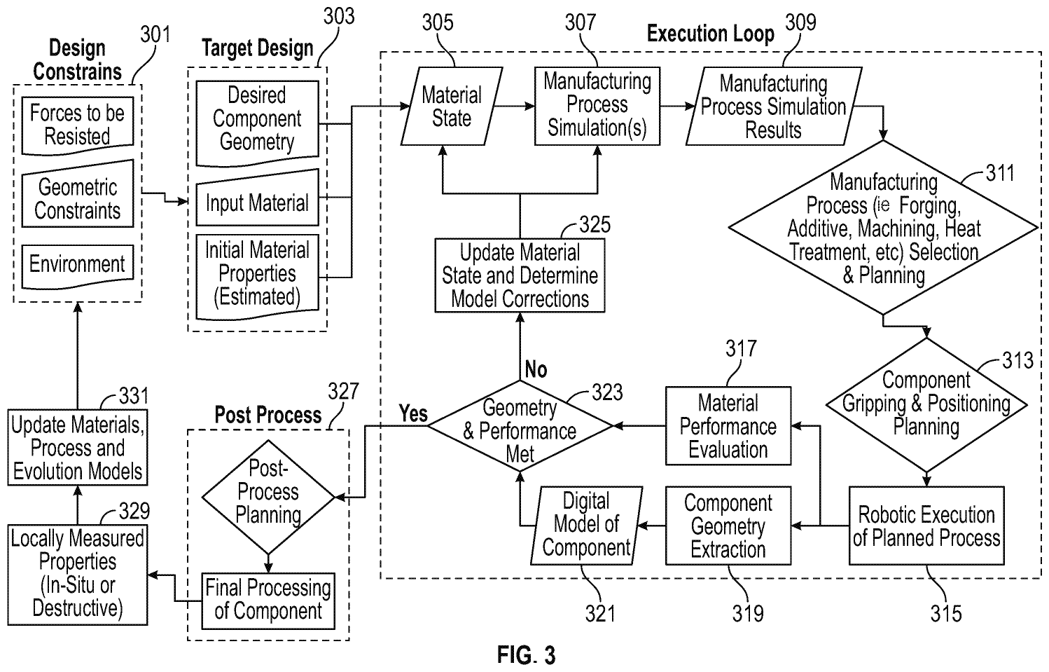

The Auto-Fab 100 as described herein provides the following advantages. First, the Auto-Fab 100 may provide for the concurrent design of components or products as well as the manufacturing process to create such components. A suitable method for the concurrent design of components and manufacturing processes is described further with respect to FIGS. 2 and 3.

Second, the Auto-Fab 100 allows for the easy transfer of manufactured components from one manufacturing process to another, enabling for a new type of manufacturing. For example, a group of Auto-Fabs 100 may be available at a location. A first Auto-Fab 100 may produce a first component using a first manufacturing process. A second Auto-Fab 100 may use the first component as material in a second process that is used to create a second component, and so forth. Note that depending on the embodiment, the Auto-Fabs 100 may be at different locations, allowing for a first Auto-Fab 100 at a first location where first materials are available to generate a first component, and a second Auto-Fab 100 at a second location where second materials are available to generate a second component. The first and second components may then be used by a third Auto-Fab 100 as materials to generate a third component. In many cases the Auto Fabs 100 may be very similar to existing manufacturing processes. They are distinguished by the integration into a single network that considers the design of component and process with the execution of the manufacturing processes. This may include autonomous control of individual processes, including error correction, and variations in process sequence.

Third, the Auto-Fabs 100 may be designed to incorporate artificial intelligence to allow the corresponding robotic components to control currently high-touch processes, assess routes to manufacturing a product, and select options on the pareto front. These Auto-Fabs 100 with AI may learn, improve, and share information with the other Auto-Fabs 100. The core to learning is the collection of in-process and component performance data. This data is used to train machine learning models (neural networks, deep learning, etc.) with training sets. These training data sets will allow improved system control.

Fourth, the Auto-Fabs 100 may be used as hands-on learning tools for training and education. For example, an Auto-Fab 100 may be placed in a location such as a community college. The students may use the Auto-Fab 100 to learn about manufacturing by observing the operation of the Auto-Fab 100 when deigning components and by creating novel processes for the Auto-Fab 100 to use to create new or existing components.

Fifth, the including of numerically controlled deformation and in particular improving the structure of materials with defects, such as from additive manufacturing or casting, processing in the Auto-Fab 100 allows for improvements to many existing manufacturing chains. Numerically controlled deformation addresses urgent needs in high-value supply chains by replacing closed-die forgings that can take months to obtain with incrementally formed open-die forgings for rapid access to new or replacement forgings. Very large parts could also be forged incrementally opening possibilities for new alloys that are not available in ingot form, for example grading composition or enabling new compositions such as those known as high entropy or multi-principal element alloys.

Sixth, the Auto-Fab 100 and the manufacturing processes described herein may allow for the solution of many manufacturing processes that are currently difficult. Examples include: chemically heterogeneous components can be made by the Auto-Fab 100 using deposition, hot working, and finishing; aircraft components can be made by the Auto-Fab 100 abiding by known thermomechanical processing recipes in given locations of a component; ultra large components such as hulls, keels, bulkheads, marine propellors, tie rods, and air craft stringers, for example, can be fabricated by the Auto-Fab 100 via incremental deformation; medical components including bone fixation plates and mandibular components can be fabricated by the Auto-Fab 100 via agile cutting of sheet of uniform thickness and then shaping to a patient's anatomy; the Auto-Fab 100 may use local deformation to give local properties to emerging materials systems such as high entropy alloys; the Auto-Fab 100 may also use local deformation to correct issues with porosity or coarse phases in components created by additive manufacturing or casting; and the Auto-Fab 100 may use deformation of bound powders to create pre-forms that may be shaped and optimized. Other manufacturing processes may be used by the Auto-Fab 100.

FIG. 2 is an illustration of a method 200 for a concurrent component creation and manufacturing process design. The method may be used to concurrently design a component and a manufacturing process to be executed by one or more Auto-Fabs 100 to manufacture a desired component or object.

At 201, design constraints are received. The design constraints may be constraints for a component that is to be manufactured by a device such as an Auto-Fab 100. The constraint may specify qualities of the component such as dimensions, stiffness, mechanical strength, fatigue resistance, fracture resistance, color, weight, and materials. The constraints may further include tolerances that may control how close the qualities of the manufactured component must be to the provided constraints. The design constraints may include minimum and maximum qualities such as weight and may identify one or more suitable materials. The constraints may be received by a computing device including computing component 115 of the Auto-Fab 100.

At 202, the component is designed. The component may be designed based on the received design constraints, available tools, and available materials. The component may be designed by a same or different computing device that received the design constraints. The tools and available materials may be the tools and materials that are available to the Auto-Fab 100. As may be appreciated, each Auto-Fab 100 may have different available tools and materials based on its location (e.g., outer space vs. earth location). Depending on the embodiment, when selecting the materials for the component, the computing device may favor those materials specified by the design constraints and may only substitute materials when the specified materials are unavailable at the location of the Auto-Fab 100. Key performance attributes for the component may be predicted.

At 203, a manufacturing process is designed for the component. The manufacturing process may be designed by the Auto-Fab 100 using the tools that are available at the Auto-Fab 100. Any system or method for designing a manufacturing process may be used. The manufacturing process may be designed concurrently with the component. Key performance attributes for the process may be predicted.

At 204, the process is executed. The designed manufacturing process is executed by the Auto-Fab 100 using the materials available to the Auto-Fab 100. The result of the executed manufacturing process is the component.

At 205, data from the process is retrieved. The data may be associated with a data stream that is generated by the Auto-Fab 100 during the manufacturing process. The data may describe the characteristics (e.g., dimensions, weight, and materials) of the completed component.

At 206, the data is examined to determine component quality. In particular, the data may be examined to determine if the characteristics of the completed component are within the tolerances provided in the design constraints at 201. Whether or not the characteristics of the manufactured component are within the tolerances may be provided as feedback to the manufacturing design at 203. The feedback may be used to update the models used to predict the performance of the manufactured component and manufacturing process.

FIG. 3 is an illustration of a method 300 that takes design constraints (i.e., component geometry and spatially resolved performance goals) that are digitally recorded and creates a component via a primary process such as numerically controlled deformation, coupled with additional manufacturing processes (i.e., casting, additive, machining, and heat treatment). The method 300 may be closed loop in that errors will be corrected through an incremental approach to achieving the desired geometry and performance goals. Typically, the initial process plan will be developed by numerical physics-based simulations often with human design decisions. The simulation techniques may include solid mechanics or finite element analysis codes performance and physics or process codes to simulate the process. This may lead to materials for structure, which may lead to materials properties to simulate the performance of the component based on design and manufacturing decisions. As data is amassed, these physics-based codes may be increasingly replaced with machine-learning models. The method 300 may be implemented using a general-purpose computing device such as the computing device 400 illustrated with respect to FIG. 4, for example.

As shown, the method 300 begins at 301 when design constraints for a component to be fabricated are created. In the example shown, the constraints include forces to be resisted (e.g., how much static and dynamic forces must the component be able to support), geometric constraints (e.g., the maximum or minimum dimensions of the component), and environmental constraints (e.g., expected environmental temperature, humidity, and gravity). Other design constraints may be supported.

At 303, the target design of the component is created based on the design constraints. In the example shown, the target design includes desired component geometry (e.g., what the actual dimensions of the competed component should be), the input material (e.g., what materials will actually be used to generate the component), and the estimated input material properties (e.g., the estimated properties of the materials that will be used such as density, mass, and cost). Depending on the embodiment, the target design may be determined based on the materials available at the location(s) of the Auto-Fab(s) 100 that are expected to manufacture the component.

The process in 303 may be considered in consort with a range of varied materials and manufacturing processes. For example, for a given component, varied designs and processes are considered including, possibly, the use of a single piece casting, a welded assembly, a forged component, and combinations thereof, including forging a welded or cast component. This concurrent design of component and manufacturing process, including consideration of local material properties to find a most effective component and process design is central to this process. Often several viable designs and manufacturing pathways are possible.

After the design constraints and the target design are created, the execution loop may begin. The execution loop may be an iterative feedback loop where various manufacturing processes for the component are determined and tested. The results of each manufacturing process is evaluated and used to make adjustments to the manufacturing process.

The execution loop of the method 300 may begin at 305 where a material state is evaluated. The material state may be the state of the materials that will be used to construct the component according to the target design. The state of the materials may be specific to the materials that are available at the location of the Auto-Fab 100 that will be used to fabricate the component. For example, materials properties can be effectively changed by metallurgical processes such as strain hardening, recrystallization, and deformation-induced residual stress. The state of the material can be assessed by numerous methods including measurement of forming forces, local hardness measurements, estimation from strain and temperature path and more advanced techniques such as x-ray diffraction. This state evolution may be tracked and methods from physical metallurgy may be used to optimize the local state of the material. Often this will involve manipulating the temperature and strain path including amount of effective strain and principal directions, or directions of maximum extension.

At 307, a plurality of manufacturing processes that could be used to manufacture the component are simulated. The one or more processes may be the processes that are available to the Auto-Fab 100 that will fabricate the component. The plurality of processes may be the processes that are known to the Auto-Fab 100 (i.e., stored in the computing component 115) and the tools that are available to the Auto-Fab 100 (i.e., the tools that are part of the tool component 120). Any method for simulating a manufacturing process may be used.

At 309, results of the plurality of manufacturing process simulations are received. The results may include a variety of information such as the estimated speed of each process, whether each process was successfully completed, and the predicted quality of the component generated by each process. In many ways this may approximate the process that a human blacksmith uses. Depending on multiple inputs such as material temperature, surface condition, and even the sound of hammer blows, the smith may decide to heat, hammer harder, softer or in different locations. This is based on human learning. The current mechanical autonomous system will function similarly, taking in data and making decisions as to how to treat the material with its state in mind as well.

At 311, based on the results of the simulation, a manufacturing processes from among the one or more simulated manufacturing processes is selected. As may be appreciated, the creation of a component may require multiple steps (e.g., welding, forming, and painting). Preliminary process paths may be determined in the part and process design stage. This may be modified based on process data during execution.

At 313, component gripping and position planning are determined. The component gripping and position planning may be for the robot component 110 (e.g., the robotic arms) and may include the various positions and placements of the component as it moved through the selected manufacturing process. There may be a standard base for the robot component that allows rapid definition of the component geometry. This may be fixed to a base or on a robot. Other tools may be end effectors on one or more robots.

At 315, robotic execution of the planned process is performed. The robotic component 110 may perform the process using the materials and tools available to the Auto-Fab 100. The result of the execution of the planned process is the fabricated component.

At 317, the performance of the materials used to create the component are evaluated. The evaluation may be based on monitoring of the material state and estimates will be made if it meets desired requirements such as the forces to be resisted.

At 319, the geometries of the generated component are extracted. The geometries may include the physical dimensions of the fabricated component. Any method for extracting geometries from a component may be used.

At 321, a digital model of the fabricated component is generated. The digital model may be generated based on the extracted geometries. Any method for generating a digital model may be used.

At 323, whether the determined geometry and material performance met the requirements of the target design and design constraints. If not, the method 300 may go to 325 and the execution loop may continue. Otherwise, the method 300 may continue at 327 and may exit the execution loop.

At 325, the material state is updated and model corrections are determined and executed. For example, shape can be improved by deformation, and additional cold work could be added to improve local hardness or residual stresses. After the material state is updated or improved and component dimensional corrections are determined, the method 300 may return to 307 where the plurality of manufacturing processes are simulated using the new material state and corrected model and the execution loop repeats resulting in a new component being generated. The execution loop may continue to repeat until its determined that the geometry and performance are met by the generated component at 323.

At 327, post processing of the component is performed. This may include heat treating, surface finishing, residual stress optimization via bending or peening. Other operations may be included.

At 329, properties of the component are estimated by through knowledge of material state and its known dimensions. Prior data and experience that is acquired and used in machine learning routines will improve the accuracy of these estimates. Step 329 may be a final check to ensure that the final component meets design intents.

At 331, materials, the process, and the evolution model are updated. At 331, data is acquired from the current process, integrated with other available similar operations and used to improve the machine learning models. Physics-based models can also be used.

With reference to FIG. 4, an exemplary system for implementing aspects described herein includes a computing device, such as computing device 400. In its most basic configuration, computing device 400 typically includes at least one processing unit 402 and memory 404. Depending on the exact configuration and type of computing device, memory 404 may be volatile (such as random access memory (RAM)), non-volatile (such as read-only memory (ROM), flash memory, etc.), or some combination of the two. This most basic configuration is illustrated in FIG. 4 by dashed line 406.

Computing device 400 may have additional features/functionality. For example, computing device 400 may include additional storage (removable and/or non-removable) including, but not limited to, magnetic or optical disks or tape. Such additional storage is illustrated in FIG. 4 by removable storage 408 and non-removable storage 410.

Computing device 400 typically includes a variety of computer readable media. Computer readable media can be any available media that can be accessed by the device 400 and includes both volatile and non-volatile media, removable and non-removable media.

Computer storage media include volatile and non-volatile, and removable and non-removable media implemented in any method or technology for storage of information such as computer readable instructions, data structures, program modules or other data. Memory 404, removable storage 408, and non-removable storage 410 are all examples of computer storage media. Computer storage media include, but are not limited to, RAM, ROM, electrically erasable program read-only memory (EEPROM), flash memory or other memory technology, CD-ROM, digital versatile disks (DVD) or other optical storage, magnetic cassettes, magnetic tape, magnetic disk storage or other magnetic storage devices, or any other medium which can be used to store the desired information and which can be accessed by computing device 900. Any such computer storage media may be part of computing device 500.

Computing device 400 may contain communication connection(s) 412 that allow the device to communicate with other devices. Computing device 400 may also have input device(s) 414 such as a keyboard, mouse, pen, voice input device, touch input device, etc. Output device(s) 416 such as a display, speakers, printer, etc. may also be included. All these devices are well known in the art and need not be discussed at length here.

It should be understood that the various techniques described herein may be implemented in connection with hardware components or software components or, where appropriate, with a combination of both. Illustrative types of hardware components that can be used include Field-programmable Gate Arrays (FPGAs), Application-specific Integrated Circuits (ASICs), Application-specific Standard Products (ASSPs), System-on-a-chip systems (SOCs), Complex Programmable Logic Devices (CPLDs), etc. The methods and apparatus of the presently disclosed subject matter, or certain aspects or portions thereof, may take the form of program code (i.e., instructions) embodied in tangible media, such as floppy diskettes, CD-ROMs, hard drives, or any other machine-readable storage medium where, when the program code is loaded into and executed by a machine, such as a computer, the machine becomes an apparatus for practicing the presently disclosed subject matter.

Although exemplary implementations may refer to utilizing aspects of the presently disclosed subject matter in the context of one or more stand-alone computer systems, the subject matter is not so limited, but rather may be implemented in connection with any computing environment, such as a network or distributed computing environment. Still further, aspects of the presently disclosed subject matter may be implemented in or across a plurality of processing chips or devices, and storage may similarly be effected across a plurality of devices. Such devices might include personal computers, network servers, and handheld devices, for example.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

What is claimed:

1. A method for manufacturing a component comprising:
receiving design constraints for a component to be manufactured by a computing device;
determining available tools and available materials at a location by the computing device;
determining one for more designs for the component based on the design constraints, determined available tools, and determined available materials by the computing device;
determining one or more manufacturing processes for the component based on the determined design and determined available tools by the computing device;
predicting attributes of the determined one or more designs and the determined one or more manufacturing processes using one or more models by the computing device;
choosing one or more combinations of the determined designs and manufacturing processes to create the component based on the predicted attributes by the computing device;
executing the chosen manufacturing processes to generate the component according to the chosen design by the computing device;
receiving data generated during the chosen manufacturing processes about the generated component by the computing device;
determine actual attributes of the generated component and the chosen manufacturing processes from the received data by the computing device; and
updating the one or more models based on the predicted attributes and the actual attributes by the computing device.

2. The method of claim 1, further comprising certifying a component or manufacturing process using the updated one or more models.

3. The method of claim 1, wherein the manufacturing process is an automated manufacturing process and includes one or more of deformation, casting, machining, additive manufacturing and welding.

4. The method of claim 1, wherein the manufacturing process is performed by an Auto-Fab system, and the location is the location of the Auto-Fab system.

5. The method of claim 1, wherein the manufacturing process is performed by a virtual Auto-Fab system, and the component may be shipped from one location to another during manufacturing.

6. The method of claim 4, wherein the Auto-Fab system comprises a housing, the determined available tools, and a robotics component adapted to perform the manufacturing process using the determined available tools, wherein the Auto-Fab system uses standard component bases that aid in component positioning and provide rapid transfer from one location to another, and further wherein the component bases include compliant, break-away, or deformable links.

7. The method of claim 1, wherein the component is a medical device tailored to conform to a patient's anatomy.

8. The method of claim 1, wherein the design is topologically optimized within manufacturing constraints to meet structural constraints such as strength, stiffness, fracture resistance, fatigue resistance, corrosion resistance, and corrosion fatigue resistance.

9. A system comprising:
at least one computing device; and
a computer-readable medium storing computer-executable instructions stored thereon that when executed by the at least one computing device, cause the at least one computing device to:
receive design constraints for a component to be manufactured;
determine available tools and available materials at a location;
determine a design for the component based on the design constraints, determined available tools, and determined available materials;
determine a manufacturing process for the for the for the component based on the determined design and determined available tools;
execute the manufacturing process to generate the component;
receive data generated during the manufacturing process about the generated component; and
based on the data generated during the manufacturing process, determine that the generated component does not satisfy the received design constraints; and
in response to the determination that the generated component does not satisfy the received design constraints, adjust the determined manufacturing process.

10. The system of claim 9, further comprising computer-executable instructions stored thereon that when executed by the at least one computing device, cause the at least one computing device to: in response to the determination that the generated component does not satisfy the received design constraints, adjust the determined design by the computing device.

11. The system of claim 9, wherein the manufacturing process is an automated manufacturing process and includes one or more of deformation, casting, machining, additive manufacturing, and welding.

12. The system of claim 8, wherein the manufacturing process is performed by an Auto-Fab system, and the location is the location of the Auto-Fab system.

13. The system of claim 12, wherein the Auto-Fab system comprises a housing, the determined available tools, and a robotics component adapted to perform the manufacturing process using the determined available tools.

14. The system of claim 8, wherein the component is a medical device.

15. The system of claim 9, wherein the design constraints comprise geometric constraints, forces to be resisted by the component, and environment constraints.

16. A non-transitory computer-readable medium storing computer-executable instructions stored thereon that when executed by at least one computing device, cause the at least one computing device to:
receive design constraints for a component to be manufactured;
determine available tools and available materials at a location;
determine a design for the component based on the design constraints, determined available tools, and determined available materials;
determine a manufacturing process for the for the for the component based on the determined design and determined available tools;
execute the manufacturing process to generate the component;
receive data generated during the manufacturing process about the generated component; and
based on the data generated during the manufacturing process, determine that the generated component does not satisfy the received design constraints; and
in response to the determination that the generated component does not satisfy the received design constraints, adjust the determined manufacturing process.

17. The non-transitory computer-readable medium of claim 16, further comprising computer-executable instructions that when executed by the at least one computing device, cause the at least one computing device to: in response to the determination that the generated component does not satisfy the received design constraints, adjust the determined design by the computing device.

18. The non-transitory computer-readable medium of claim 16, wherein the manufacturing process is an automated manufacturing process and includes one or more of deformation, casting, machining, additive manufacturing and welding.

19. The non-transitory computer-readable medium of claim 16, wherein the manufacturing process is performed by an Auto-Fab system, and the location is the location of the Auto-Fab system.

20. The non-transitory computer-readable medium of claim 18, wherein the Auto-Fab system comprises a housing, the determined available tools, and a robotics component adapted to perform the manufacturing process using the determined available tools.
